(19) United States
(12) Patent Application Publication
(10) Pub. No.: US 2025/0352764 A1
(43) Pub. Date: Nov. 20, 2025

(54) SURGICAL METHOD FOR DELIVERING DRUG SOLUTION TO MYOCARDIUM

(71) Applicants: ASAHI INTECC CO., LTD., Seto-shi (JP); CUORIPS INC., Tokyo (JP)

(72) Inventors: Yoshiki SAWA, Tokyo (JP); Satoru Sumitsuji, Seto-shi (JP); Gaku Nakazawa, Tokyo (JP); Masaaki Nihonmatsu, Seto-shi (JP); Nobuyoshi Watanabe, Seto-shi (JP); Hiroshi Itou, Seto-shi (JP); Kotaro Mizukami, Seto-shi (JP); Kouichi Hasegawa, Tokyo (JP); Hiroko Sannabe, Tokyo (JP)

(73) Assignees: ASAHI INTECC CO., LTD., Seto-shi (JP); CUORIPS INC., Tokyo (JP)

(21) Appl. No.: 19/202,608

(22) Filed: May 8, 2025

(30) Foreign Application Priority Data
May 15, 2024 (JP) 2024-079378

(51) Int. Cl. A61M 25/00; A61B 17/00; A61M 25/01

(52) U.S. Cl. CPC A61M 25/0084; A61B 17/00234; A61M 25/0108; A61B 2017/00243; A61M 2025/0089

(57) ABSTRACT

A surgical method for delivering a drug solution to myocardium includes the steps of: inserting a catheter into at least one of a coronary artery and a coronary vein, the catheter having a lumen, a lateral opening connecting the lumen and outside of the catheter, and a radiopaque marker indicating a position of the lateral opening; rotating the catheter while observing the marker such that an orientation of the lateral opening becomes a predetermined orientation with respect to a treatment target site; projecting a puncture needle through the lateral opening; piercing a myocardium of the treatment target site with the puncture needle; and injecting a drug solution containing therapeutic cells or chemical compounds from the puncture needle into the myocardium.

### CROSS-REFERENCE TO RELATED APPLICATIONS

The present application claims the benefit of Japanese Patent Application No. 2024-079378 filed on May 15, 2024, the disclosures of which are incorporated by reference in their entirety.

### TECHNICAL FIELD

The present disclosure relates to a surgical method that enables a drug solution to be delivered to a human heart using a catheter.

### BACKGROUND

Regenerative treatment methods are known that regenerate myocardium whose function has been impaired by myocardial infarction or the like. For example, Japanese Patent No. 5,572,138 discloses a treatment method in which cardiomyocytes are prepared in a sheet form outside the body, and then the cell sheet is attached to the heart to promote the regeneration of myocardium. Because the heart is constantly beating, it is difficult to keep a cell sheet stably attached to the heart for long periods of time. Therefore, a treatment method that promotes the regeneration of myocardium by injection of a drug solution to the myocardium is being sought. Such a treatment method is referred to as a “drug solution injection treatment” below. In a drug solution injection treatment, a drug solution for promoting regeneration of myocardium is injected to the myocardium from at least one of a coronary artery and a coronary vein.

For example, JP 2023-048291 A discloses a catheter that can be used in drug solution injection treatment, which has a lumen for projecting a puncture needle. For example, JP 2004-329487 A discloses a drug solution injection device having a needle-shaped tubular body, and a drug solution supply means that supplies a drug solution to the needle-shaped tubular body.

### SUMMARY

In order to pierce the myocardium with a puncture needle from a catheter that has been inserted into a coronary artery or a coronary vein, it is preferable to provide a lateral opening in the catheter for projecting the puncture needle. In this regard, the devices described in JP 2023-048291 A and JP 2004-329487 A have a lateral opening in the catheter. In a drug solution injection treatment, a surgeon rotates and aligns the catheter such that the lateral opening of the catheter faces the myocardium, and then uses the puncture needle to pierce the myocardium. In the devices described in JP 2023-048291 A and JP 2004-329487A, there is still room for improvement with respect to the alignment of such a lateral opening and the myocardium.

The present disclosure has been made to solve at least a part of the problem described above. The present disclosure has an object of simplifying alignment between a lateral opening and the myocardium, and improving the efficiency of drug solution injection treatment procedures.

According to an aspect of the present disclosure, a surgical method for delivering a drug solution to myocardium is provided. The surgical method includes the steps of: inserting a catheter into at least one of a coronary artery and a coronary vein, the catheter having a lumen, a lateral opening connecting the lumen and outside of the catheter, and a radiopaque marker indicating a position of the lateral opening; rotating the catheter while observing the radiopaque marker such that an orientation of the lateral opening becomes a predetermined orientation with respect to a treatment target site; projecting a puncture needle through the lateral opening; piercing a myocardium with the puncture needle; and injecting a drug solution containing therapeutic cells or chemical compounds from the puncture needle into the myocardium.

### BRIEF DESCRIPTION OF DRAWINGS

FIG. 1 is an explanatory diagram illustrating a configuration of a medical system.

FIG. 2 is an explanatory diagram illustrating a cross-sectional configuration of a first shaft of a first catheter.

FIG. 3 is a transverse cross-sectional view along line A 1-A 1 in FIG. 2.

FIG. 4 is a transverse cross-sectional view along line A 2-A 2 in FIG. 2.

FIG. 5 shows the shape of a marker when viewed from the −Z axis direction side.

FIG. 6 shows the shape of a marker when viewed from the −Y axis direction side.

FIG. 7 shows the shape of a marker when viewed from the +Z axis direction side.

FIG. 8 is an enlarged view of the vicinity of a marker of the first catheter 1.

FIG. 9 is an image of a marker in the orientation shown in FIG. 5 obtained under an X-ray image.

FIG. 10 is an image of a marker in the orientation shown in FIG. 6 obtained under an X-ray image.

FIG. 11 is an image of a marker in the orientation shown in FIG. 7 obtained under an X-ray image.

FIG. 12 is an explanatory diagram showing the shape of a marker under an X-ray image.

FIG. 13 is a transverse cross-sectional view of a first shaft in a section SE.

FIG. 14 is an explanatory diagram illustrating a configuration of a second catheter.

FIG. 15 is a transverse cross-sectional view along line B1-B1 in FIG. 14.

FIG. 16 is a transverse cross-sectional view along line B2-B2 in FIG. 14.

FIG. 17 is a transverse cross-sectional view along line B3-B3 in FIG. 14.

FIG. 18 is an enlarged view of a connector of a second catheter.

FIG. 19 is a longitudinal cross-sectional view of a connector.

FIG. 20 is a longitudinal cross-sectional view of a connector to which a second shaft has been attached.

FIG. 21 is a diagram describing a drug solution.

FIG. 22 is a diagram showing a state in which a drug solution injection treatment is being performed on the heart.

FIG. 23 is a diagram showing a state where a first catheter has been delivered to a treatment target site.

FIG. 24 is a diagram showing a state of a first catheter in which a lateral opening is facing the myocardium.

FIG. 25 is a diagram showing a state where a balloon catheter is being delivered.

FIG. 26 is a diagram showing a state where a stylet wire is being inserted through a second catheter.

FIG. 27 is a diagram showing a state where a second catheter is being delivered.

FIG. 28 is a diagram showing a state where a buffer solution is being injected into a second catheter.

FIG. 29 is a diagram showing a state where a drug solution is being injected into a second catheter.

FIG. 30 is a diagram showing a state where the myocardium is being pierced.

FIG. 31 is a diagram showing a state where a drug solution is being injected into the myocardium.

FIG. 32 is a diagram describing a drug solution according to a second embodiment.

FIG. 33 is a diagram showing the shape of a marker when a first catheter according to a third embodiment is viewed from the −Y axis direction.

### DESCRIPTION OF EMBODIMENTS

### First Embodiment

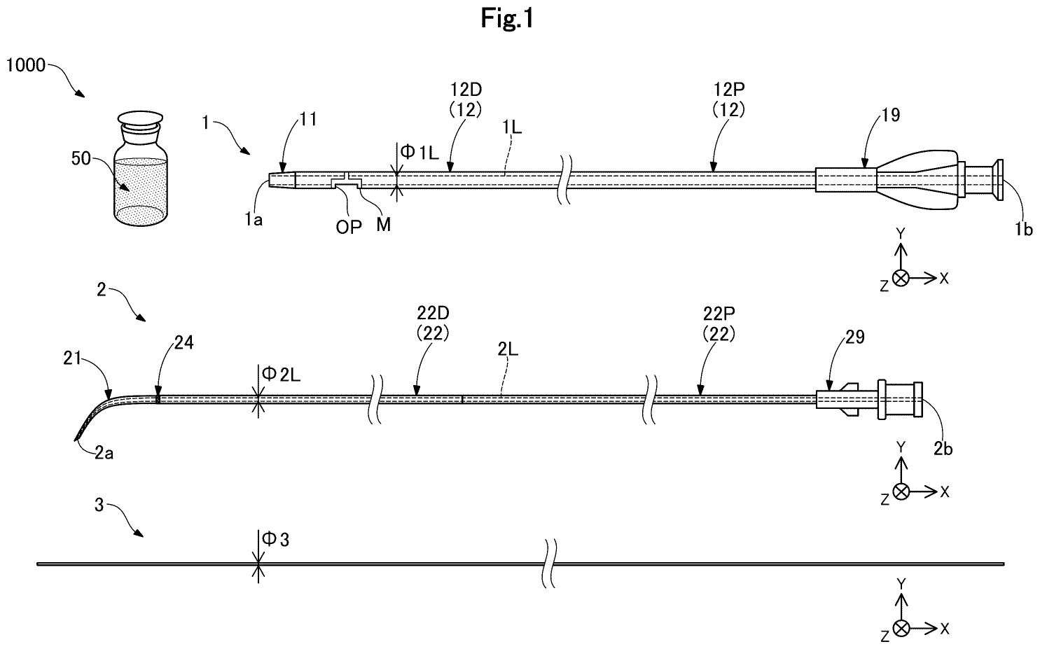

FIG. 1 is an explanatory diagram illustrating a configuration of a medical system 1000. The medical system 1000 according to the present embodiment includes a first catheter 1, a second catheter 2, a stylet wire 3, and a drug solution 50. The medical system 1000 is a system used for a drug solution injection treatment. A drug solution injection treatment refers to a treatment method that promotes regeneration of myocardium by injection of a drug solution containing therapeutic cells or chemical compounds from at least one of a coronary artery and a coronary vein into the myocardium. The target site of a drug solution injection treatment is also referred to as a “treatment target site”.

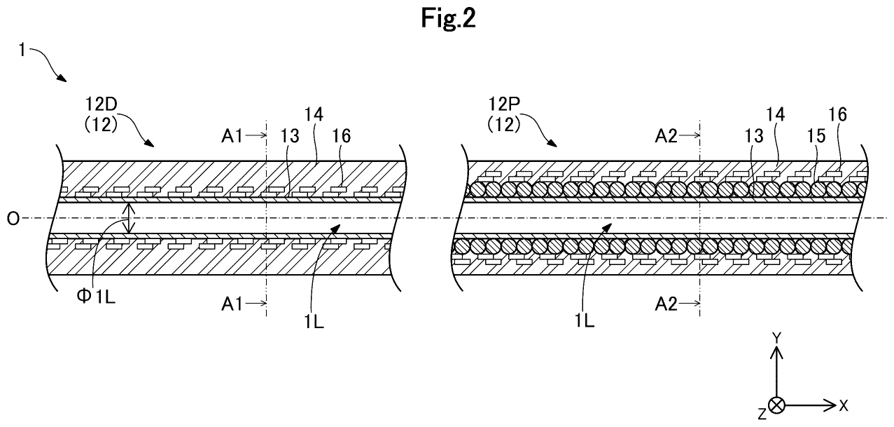

For convenience of the description, FIG. 1 includes parts where the components are illustrated with a relative size ratio which is different from the actual relative size ratio. FIG. 1 includes parts in which portions of the components are exaggerated. FIG. 1 illustrates X, Y, and Z axes that are perpendicular to each other. The X axis corresponds to the longitudinal direction of each of the first catheter 1, the second catheter 2, and the stylet wire 3. The X axis is also referred to as the axial direction. The Y axis corresponds to the height direction of each of the first catheter 1, the second catheter 2, and the stylet wire 3. The Z axis corresponds to the width direction of each of the first catheter 1, the second catheter 2, and the stylet wire 3. The left side of FIG. 1 is referred to the “distal end side” of each device and each component. The right side of FIG. 1 is referred to as the “proximal end side” of each device and each component. The left side of FIG. 1 is the −X axis direction. The right side of FIG. 1 is the +X axis direction. Of the two ends of each device and each component in the longitudinal direction, the end positioned on the distal end side is referred to as the “distal end”. Of the two ends of each device and each component in the longitudinal direction, the other end positioned on the proximal end side is referred to as the “proximal end”. The distal end and the vicinity thereof are referred to as a “distal end portion”. The proximal end and the vicinity thereof are referred to as a “proximal end portion”. The distal end side is inserted into a living body, and the proximal end side is operated by a surgeon, such as a physician. These points also apply to FIG. 2 and subsequent diagrams. In the present embodiment, the terms “same” and “equal” have the meaning of being approximately the same, allowing for variations due to manufacturing errors and the like. In the present embodiment, the term “constant” also includes the meaning of approximately constant, allowing for variations due to manufacturing errors and the like.

The first catheter 1 is a catheter for delivering the second catheter 2 to a treatment target site. The first catheter is also referred to as a delivery catheter. As shown in FIG. 1, the first catheter 1 includes a distal end tip 11, a first shaft 12, a marker M, and a catheter connector 19. The first catheter 1 is a single lumen catheter having a single first lumen 1L. The first catheter 1 has a lateral opening OP.

The distal end tip 11 is provided on the distal end portion of the first shaft 12, and moves inside the blood vessel ahead of the other members. The distal end tip 11 is a cylindrically-shaped member having an outer diameter that gradually decreases from the proximal end side toward the distal end side. The distal end of the distal end tip 11 is formed having a distal end opening 1a. The distal end opening 1a is an opening for inserting other devices into the first catheter 1. Examples of other devices include guide wires for delivery referred to as workhorse wires.

The first shaft 12 includes the first lumen 1L in the interior, and is a tubular body having an elongated outer shape. The first shaft 12 includes a distal end side shaft 12D and a proximal end side shaft 12P. A lateral surface of the distal end side shaft 12D is provided with the lateral opening OP. The lateral opening OP is a through hole that is formed in a lateral surface of the distal end side shaft 12D of the first catheter 1, and is a through hole that connects the first lumen 1L and the outside of the catheter. The lateral opening OP is an opening for projecting the distal end portion of the second catheter 2 from the first catheter 1. The lateral opening OP has a substantially rectangular shape when viewed from the −Y axis direction side. The details of the lateral opening OP will be described later. The lateral opening OP may have a shape that is different from a substantially rectangular shape when viewed from the−Y axis direction. The different shape may be, for example, a circular shape, a square shape, or a polygonal shape. Of the distal end side shaft 12D, the vicinity of the lateral opening OP is provided with a radiopaque marker M. The marker M is a guide for the surgeon to confirm the orientation of the lateral opening OP under an X-ray image. An X-ray image is also referred to as an angiographic image. The details of the marker M will be described later. The distal end tip 11 is fixed to the distal end portion of the distal end side shaft 12D. The catheter connector 19 is fixed to the proximal end portion of the proximal end side shaft 12P.

The catheter connector 19 is attached to the proximal end portion of the first shaft 12, and makes it easier for the surgeon to grip the device. The catheter connector 19 is a substantially cylindrical member provided with a pair of wings. The proximal end of the catheter connector 19 is formed having a proximal end opening 1b. The proximal end opening 1b is an opening for inserting the second catheter 2 and other devices into the first catheter 1. Examples of other devices include workhorse wires.

As shown by the dashed lines in FIG. 1, the distal end tip 11, the first shaft 12, and the catheter connector 19 are formed having the first lumen 1L that communicates between the interior of each portion along the longitudinal direction of the first catheter 1. The first lumen 1L is a lumen into which the second catheter 2 and other devices are inserted. The inner diameter Φ1L of the first lumen 1L can be arbitrarily determined as long as the inner diameter Φ1L is larger than the outer diameter of the second shaft 22 of the second catheter 2. The proximal end of the first lumen 1L communicates with the outside from the proximal end opening 1b. The distal end of the first lumen 1L communicates with the outside from the distal end opening 1a. The distal end portion of the first lumen 1L communicates with the outside from the lateral opening OP.

The distal end tip 11 can be made of a flexible resin material, such as a polyurethane elastomer. The distal end tip 11 may be made of a radiopaque resin material or metal material. For example, when a radiopaque resin material is used, the distal end tip 11 can be formed by mixing a radiopaque material such as bismuth trioxide, tungsten, or barium sulfate with a polyamide resin, a polyolefin resin, a polyester resin, a polyurethane resin, a silicon resin, a fluorine resin, or the like. For example, when a radiopaque metal material is used, the distal end tip 11 can be formed using at least one of gold, platinum, and tungsten. The distal end tip 11 may be made of an alloy containing at least one of gold, platinum, and tungsten. The first shaft 12 and the catheter connector 19 can be formed from known materials such as a nylon resin, a polyolefin, a polyester, a thermoplastic resin, a polyamide elastomer, a polyolefin elastomer, a polyurethane elastomer, silicone rubber, and latex rubber. Examples of nylon resins include polyamide. Examples of polyolefins include polyethylene, polypropylene, and ethylene-propylene copolymers. Examples of polyesters include polyethylene terephthalate. Examples of thermoplastic resins include polyvinyl chloride, ethylene-vinyl acetate copolymers, crosslinked ethylene-vinyl acetate copolymers, and polyurethane.

The second catheter 2 is a catheter for injecting a drug solution into the myocardium. The second catheter 2 is also referred to as a needle catheter. As shown in FIG. 1, the second catheter 2 includes a puncture needle 21, a second shaft 22, a needle marker 24, and a connector 29. The second catheter 2 is a single lumen catheter having a single second lumen 2L. The puncture needle 21 is fixed to the distal end of the second catheter 2.

The puncture needle 21 is a hollow puncture needle attached to the distal end of the second shaft 22. The puncture needle 21 is also simply referred to as a “needle”. The puncture needle 21 has an outer diameter that gradually decreases from the proximal end side toward the distal end side. The distal end portion of the puncture needle 21 has a sharp shape to make it easy to pierce body tissue. The puncture needle 21 is curved in one specific direction. In the illustrated example, the puncture needle 21 is curved in the −Y axis direction. The inner cavity of the puncture needle 21 constitutes a portion of the second lumen 2L. The distal end of the puncture needle 21 is formed having a distal end opening 2a. The distal end opening 2a is an opening used to project the stylet wire 3 when the second catheter 2 is delivered, and is used to discharge the drug solution when the drug solution is injected by the second catheter 2. The puncture needle 21 may be integrally formed with the second shaft 22.

The second shaft 22 includes the second lumen 2L in the interior, and is a tubular body having an elongated outer shape. The second shaft 22 includes a distal end side shaft 22D and a proximal end side shaft 22P. The distal end of the distal end side shaft 22D is provided with a radiopaque needle marker 24. The needle marker 24 is a guide for the surgeon to confirm the position of the puncture needle 21 under an X-ray image. The needle marker 24 has an annular shape that surrounds the entire circumference of the distal end side shaft 22D. The needle marker 24 may have an arbitrary shape that is different from an annular shape, or may be omitted. The needle marker 24 can be formed of a radiopaque resin material or metal material. The puncture needle 21 is fixed to the distal end portion of the distal end side shaft 22D. The connector 29 is fixed to the proximal end portion of the proximal end side shaft 22P.

The connector 29 is attached to the proximal end portion of the second shaft 22, and makes it easier for the surgeon to grip the device. The connector 29 is also used when the surgeon introduces the buffer solution or the drug solution into the second lumen 2L. The connector 29 is a substantially cylindrical member provided with a pair of wings. The details of the connector 29 will be described later. The proximal end of the connector 29 is formed having a proximal end opening 2b. The proximal end opening 2b is an opening for inserting the stylet wire 3 and other devices into the second catheter 2. Examples of other devices include syringes. The connector 29 is formed of a known resin material.

As shown by the dashed lines in FIG. 1, the puncture needle 21, the second shaft 22, and the connector 29 are formed having the second lumen 2L that communicates between the interior of each portion along the longitudinal direction of the second catheter 2. The second lumen 2L is a lumen for inserting the stylet wire 3. The second lumen 2L is a lumen through which the buffer solution and the drug solution flows. The inner diameter Φ2L of the second lumen 2L can be arbitrarily determined as long as the inner diameter Φ2L is larger than the outer diameter Φ3 of the stylet wire 3. The distal end of the second lumen 2L communicates with the outside from the distal end opening 2a. The proximal end of the second lumen 2L communicates with the outside from the proximal end opening 2b.

The stylet wire 3 is a wire for protecting the first catheter 1 and the second catheter 2 when the medical system 1000 is used, and for making it easier to deliver the second catheter 2 by imparting rigidity to the second catheter 2. The stylet wire 3 includes a core wire and a coil. In FIG. 1, the core wire and the coil are omitted for convenience of the illustration.

The core wire is a cylindrical member having an elongated outer shape. The core wire may be configured having a constant outer diameter, or may be configured having an outer diameter that decreases from the proximal end side to the distal end side. The coil is formed by winding wires into a spiral shape. The coil is arranged so as to surround a part of the distal end side of the core wire, and is fixed to the core wire. The coil may be arranged surrounding the entire core wire from the distal end to the proximal end. The outer diameter Φ3 of the coil is set to the outer diameter of the stylet wire 3. The outer diameter Φ3 of the stylet wire 3 of the present embodiment is a value obtained by subtracting a predetermined clearance value from the smallest inner diameter of the puncture needle 21 of the second catheter 2. The smallest inner diameter of the puncture needle 21 is the inner diameter of the distal end of the puncture needle 21. In other words, the distal end of the puncture needle 21 of the second catheter 2 has an inner diameter in which the predetermined clearance value has been added to the outer diameter Φ3 of the stylet wire 3.

The core wire can be formed using, for example, at least one of a stainless steel alloy, a superelastic alloy, piano wire, a nickel-chromium alloy, a cobalt alloy, and tungsten. Examples of stainless steel alloys include SUS302, SUS304, and SUS316. Examples of superelastic alloys include nickel-titanium. The core wire may be formed of other known materials than those mentioned above. The coil can be formed using, for example, at least one of a stainless steel alloy, a superelastic alloy, a radiolucent alloy, and a radiopaque alloy. Examples of stainless steel alloys include SUS304 and SUS316. Examples of superelastic alloys include nickel-titanium alloy. Examples of radiolucent alloys include piano wire, nickel-chromium alloys, and cobalt alloys. Examples of radiopaque alloys include gold, platinum, tungsten, and alloys containing these elements. The coil may be formed of other known materials than those mentioned above.

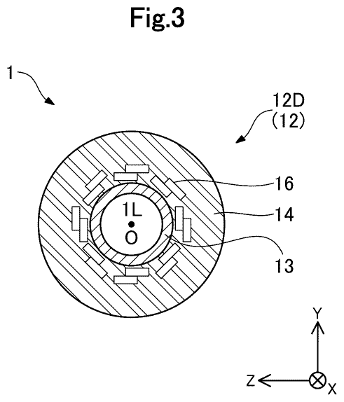

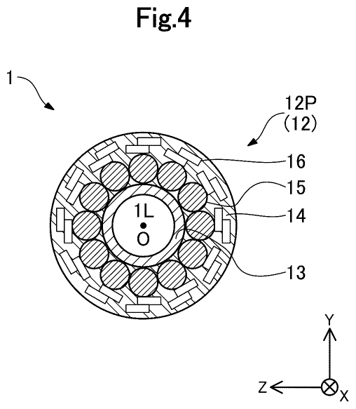

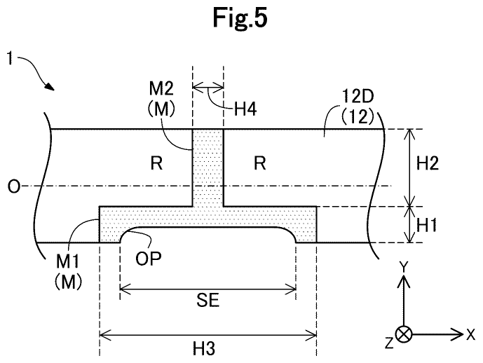

FIG. 2 is an explanatory diagram illustrating a cross-sectional configuration of the first shaft 12 of the first catheter 1. FIG. 2 is a longitudinal cross-sectional view in which the first shaft 12 is cut along the XY plane. FIG. 3 is a transverse cross-sectional view along line A1-A1 in FIG. 2. FIG. 4 is a transverse cross-sectional view along line A2-A2 in FIG. 2. The central axis of the first catheter 1 is indicated by the axis line O in FIGS. 2 to 4. The axis line O coincides with the axis passing through the center of the first shaft 12, and is the same in FIG. 5 and subsequent diagrams. The first shaft 12 includes an inner layer 13, an outer layer 14, a coil 15, and a reinforcing body 16. Hereinafter, the configuration of the first catheter 1 will be described in detail.

The inner layer 13 is an elongated tubular body provided on the inner side of the first catheter 1. The inner layer 13 defines the first lumen 1L. The outer layer 14 is provided on the outer side of the inner layer 13, and is an elongated tubular body that covers the outer periphery of the inner layer 13. The distal end portions of each of the inner layer 13 and the outer layer 14 are joined with the distal end tip 11. The rear end portions of each of the inner layer 13 and the outer layer 14 are joined with the catheter connector 19. The second catheter 2 is inserted into the first lumen 1L, which is defined by the inner layer 13. As a result, the inner layer 13 is preferably formed of a resin material having excellent sliding properties. For example, the inner layer 13 is formed of a fluorine-based polymer, polyethylene, or the like. Examples of fluorine-based polymers include PTFE, PFA, and FEP. PTFE is also referred to as polytetrafluoroethylene. PFA is also referred to as tetrafluoroethylene-perfluoroalkyl vinyl ether copolymer. FEP is also referred to as tetrafluoroethylene-hexafluoropropylene copolymer. The inner layer 13 may be made of known materials other than those described above.

The outer layer 14 can be formed of, for example, an elastomer-based resin. Examples of elastomer-based resins include PAE, TPU, and TPEE. PAE is also referred to as a polyamide-based thermoplastic elastomer. TPU is also referred to as a polyurethane thermoplastic elastomer. TPEE is also referred to as a polyester elastomer. The outer layer 14 may be made of known materials other than those described above.

In the proximal end side shaft 12P, the coil 15 is embedded in the outer layer 14 and covers the inner layer 13. The coil 15 is a multi-threaded coil formed by winding a plurality of wires into a multi-thread. The reinforcing body 16 includes a wire, and like the coil 15, is embedded in the outer layer 14 and covers the coil 15. The reinforcing body 16 is a braided body in which a wire is woven into a mesh shape. The reinforcing body 16 is a metal reinforcing body. The distal end side shaft 12D is not provided with the coil 15. In the distal end side shaft 12D, the reinforcing body 16 is embedded in the outer layer 14 and covers the inner layer 13. That is, the reinforcing body 16 is embedded in the interior of the lateral surface of both the proximal end side shaft 12P and the distal end side shaft 12D.

The coil 15 can be formed of, for example, a stainless steel alloy, a superelastic alloy, a piano wire, a radiolucent alloy, a radiopaque alloy, or known material other than those described above. Examples of stainless steel alloys include SUS304 and SUS316. Examples of superelastic alloys include nickel-titanium alloy. Examples of radiolucent alloys include nickel-chromium alloys and cobalt alloys. Examples of radiopaque alloys include gold, platinum, tungsten, and alloys containing these elements. The coil 15 may be a single-threaded coil formed by winding a single wire into a single thread. The coil 15 may be a single-threaded strand coil formed by winding a strand, in which a plurality of wires have been twisted together into a single thread. The coil 15 may be a multi-threaded strand coil formed by using a plurality of strands, in which a plurality of wires have been twisted together, and twisting the strands into a multi-thread.

The wire constituting the reinforcing body 16 can be formed of a metal material. Examples of metal materials include stainless steel alloys, nickel-titanium alloys, and radiopaque alloys. The wire constituting the reinforcing body 16 may be formed of a known metal material other than those described above. Examples of stainless steel alloys include SUS304. Examples of radiopaque alloys include gold, platinum, tungsten, and alloys containing these elements.

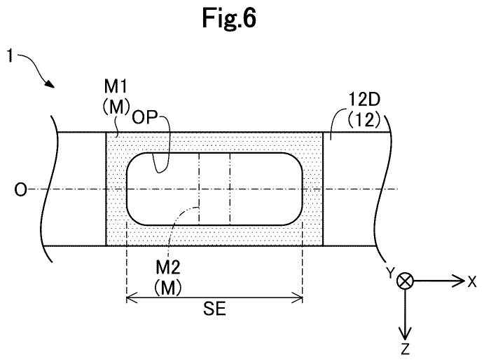

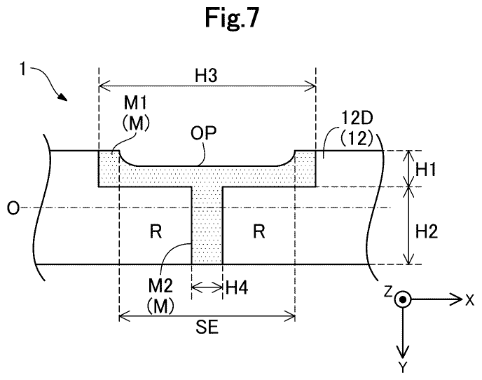

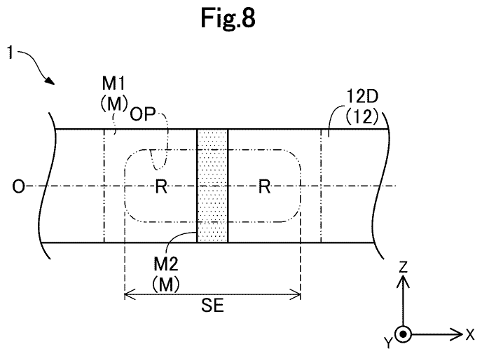

FIGS. 5 to 8 is an enlarged view of the vicinity of the marker M of the first catheter 1. FIG. 5 shows the shape of the marker M when viewed from the −Z axis direction side. FIG. 6 shows the shape of the marker M when viewed from the −Y axis direction side. FIG. 7 shows the shape of the marker M when viewed from the +Z axis direction side. FIG. 8 shows the shape of the marker M when viewed from the +Y axis direction side. In FIGS. 5 to 8, the marker M provided on the lateral surface of the distal end side shaft 12D is represented as a dotted hatching. In FIGS. 5 to 8, the part of the marker M that is not normally visible from the viewpoint in each diagram is represented by a two-dot chain line.

In FIGS. 5 and 7, the marker M is viewed from a direction perpendicular to the axis line O and the opening direction of the lateral opening OP. Hereinafter, the direction perpendicular to the axis line O and the opening direction of the lateral opening OP shown in FIGS. 5 and 7 is also referred to as the “first direction”. FIGS. 5 and 7 show the shape of the marker when viewed from the first direction side. The “opening direction” refers to the direction that the opening is facing. In FIG. 5, the first direction is the-Z axis direction, and the opening direction is the −Y direction. In FIG. 7, the first direction is the +Z axis direction, and the opening direction is the −Y direction. The shape of the marker M when viewed from the first direction side includes, as shown in FIG. 5, a shape in which the lateral opening OP is facing downward, and as shown in FIG. 7, a shape in which the lateral opening OP is facing upward. The former is referred to as the upward-facing first direction, and the latter is referred to as the downward-facing first direction.

FIG. 6 shows the shape of the marker M when viewed from the opening direction side. The opening direction is the −Y axis direction. FIG. 8 shows the shape of the marker M when viewed from the opposite side to the opening direction side. The opposite side to the opening direction side is the +Y axis direction. In FIGS. 6 and 8, the marker M is viewed from a direction perpendicular to the axis line O, and different from the first direction. Hereinafter, the direction shown in FIGS. 6 and 8 is referred to as the “second direction”. The second direction illustrated in FIGS. 6 and 8 is a direction perpendicular to the first direction, and is the +Y axis direction. The second direction can be made an arbitrary direction as long as the second direction is a direction perpendicular to the axis line O and different from the first direction.

As shown in FIGS. 5 to 8, the marker M includes a first marker portion M1 and a second marker portion M2. As shown in FIG. 6, the first marker portion M1 is provided so as to surround the outer edge of the lateral opening OP in the lateral surface of the distal end side shaft 12D. As shown in FIG. 6, the shape of the lateral opening OP when viewed from the opening direction side is a substantially rectangular shape in which the four corners are rounded. That is, the shape of the lateral opening OP when viewed from the opening direction is a shape that does not include corners.

The second marker portion M2 is provided in a residual region R which, of the lateral surface of a section SE in which the lateral opening OP is provided in the longitudinal direction of the distal end side shaft 12D, is a region in which the first marker portion M1 is not provided. The lateral surface of the section SE is the lateral surface of the distal end side shaft 12D that covers the axis line O in the section SE. In the present embodiment, the second marker portion M2 extends in the circumferential direction of the first shaft 12 in the residual region R. The circumferential direction refers to the direction around the periphery of the axis line O. In the present embodiment, the second marker portion M2 extends in the residual region R along the YZ plane, being the plane that is perpendicular to the axis line O, in the circumferential direction. Both ends of the second marker portion M2 that extend in the circumferential direction are connected to the first marker portion M1.

As shown in FIGS. 5 to 7, when the marker M is viewed from the first direction side, the length H2 of the second marker portion M2 along the opening direction is longer than the length H1 of the first marker portion M1 along the opening direction. FIGS. 5 and 7 show a perspective when the marker M is viewed from the first direction side, and the opening direction is the −Y axis direction. As shown in FIGS. 5 to 7, the length H3 of the first marker portion M1 along the longitudinal direction of the first shaft 12 is longer than the length H4 of the second marker portion M2 along the longitudinal direction of the first shaft 12.

The marker M can be formed of a radiopaque resin or metal material. For example, in a case where a resin material is used, the marker M can be formed by mixing a radiopaque material such as bismuth trioxide, tungsten, or barium sulfate with a polyamide resin, a polyolefin resin, a polyester resin, a polyurethane resin, a silicon resin, a fluorine resin, or the like. For example, when a metallic material is used, the marker M can be formed of gold, platinum, or tungsten, which are radiopaque materials, an alloy containing these elements, and the like. It is possible to use other known materials, and a joined structure may be formed by combining a plurality of materials.

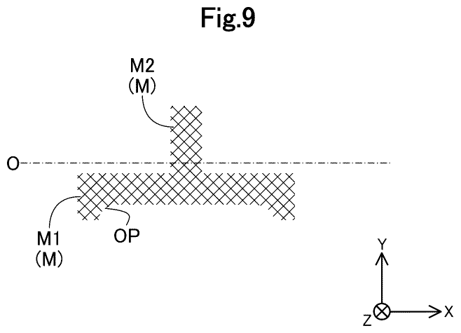

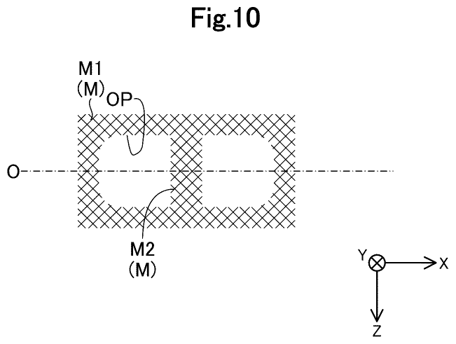

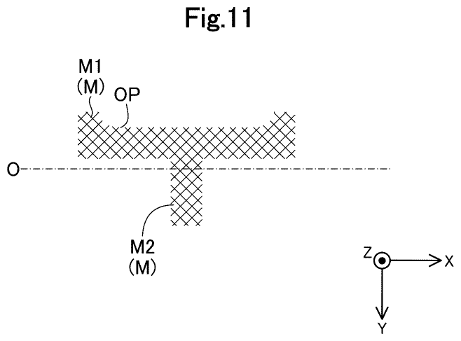

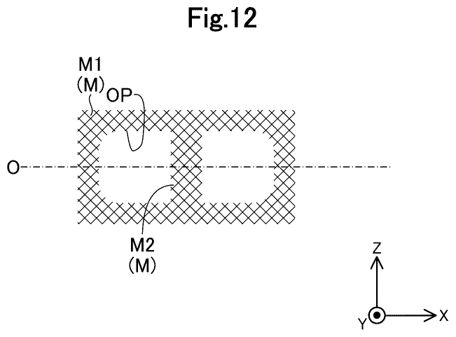

FIGS. 9 to 12 are explanatory diagrams showing the shape of the marker M under an X-ray image. FIG. 9 is an image of the marker M in the orientation shown in FIG. 5 obtained under an X-ray image. FIG. 10 is an image of the marker M in the orientation shown in FIG. 6 obtained under an X-ray image. FIG. 11 is an image of the marker M in the orientation shown in FIG. 7 obtained under an X-ray image. FIG. 12 is an image of the marker M in the orientation shown in FIG. 8 obtained under an X-ray image.

In FIGS. 9 and 11, when viewed from the first direction side, it can be clearly determined that the lateral opening OP is facing upward or downward due to the positional relationship between the first marker portion M1 and the second marker portion M2 under the X-ray image. Specifically, the orientation of the lateral opening OP can be determined depending on whether the first marker portion M1 is positioned on the upper side or lower side in the image with respect to the second marker portion M2. Under an X-ray image, the shape of the marker M viewed from the downward-facing first direction shown in FIG. 9 and the shape of the marker M viewed from the upward-facing first direction shown in FIG. 11 can be clearly distinguished. In FIGS. 10 and 12, when viewed from the second direction side, it can be clearly determined that the lateral opening OP is not facing the upper side or lower side in the image, and that the lateral opening OP is facing either the front side or the rear side in the image due to the positional relationship between the first marker portion M1 and the second marker portion M2 under the X-ray image. Specifically, the orientation of the lateral opening OP can be determined as a result of the second marker portion M2 being included on the inner side of the first marker portion M1. Under an X-ray image, the shape of the marker M viewed from the downward-facing first direction shown in FIG. 9 and the shape of the marker M viewed from the upward-facing first direction shown in FIG. 11, and the shape of the marker M from the second direction shown in FIGS. 10 and 12 are different. As a result, the surgeon can easily perform an adjustment such that the lateral opening OP faces the treatment target site side by rotating the first catheter 1, and causing the shape of the marker M under an X-ray image to change.

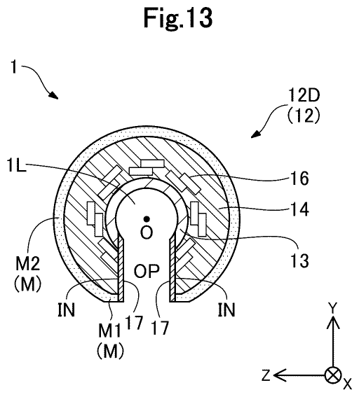

FIG. 13 is a transverse cross-sectional view of the first shaft 12 in the section SE. FIG. 13 shows a transverse cross-section in which the distal end side shaft 12D is cut along the YZ plane. As shown in FIG. 13, the first catheter 1 further includes a coating layer 17. The coating layer 17 covers an inner side surface IN of the first shaft 12 that defines the lateral opening OP, which is a through hole. The coating layer 17 prevents the reinforcing body 16, which is embedded inside the first shaft 12 around the lateral opening OP, from projecting from the inner side surface IN. In the present embodiment, the coating layer 17 is connected to the marker M. Specifically, the coating layer 17 is connected to the first marker portion M1. The coating layer 17 may, for example, be formed of the same material as the inner layer 13 or the marker M, and may be formed of an arbitrary bonding agent such as an epoxy adhesive, or a cyanoacrylic adhesive. The bonding agent used to form the coating layer 17 is preferably the same bonding agent as the bonding agent used when joining the marker M with the lateral surface of the first shaft 12.

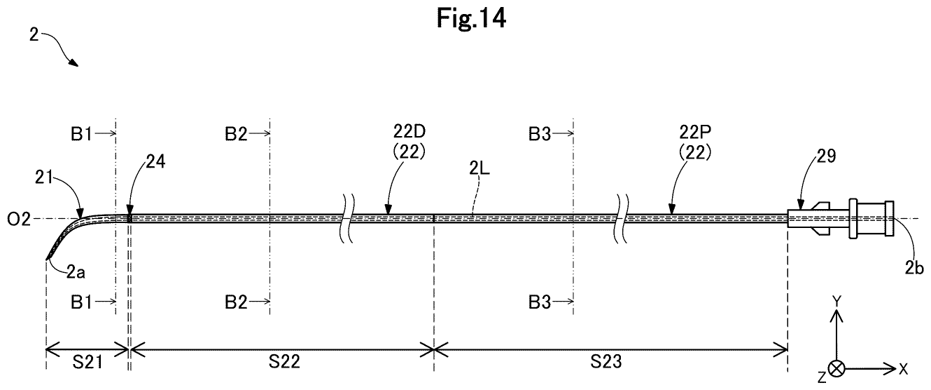

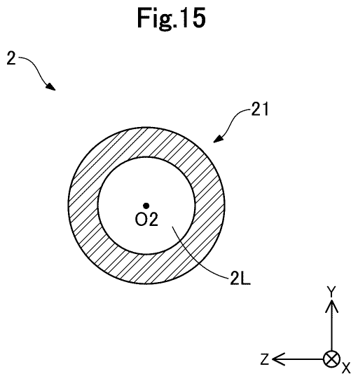

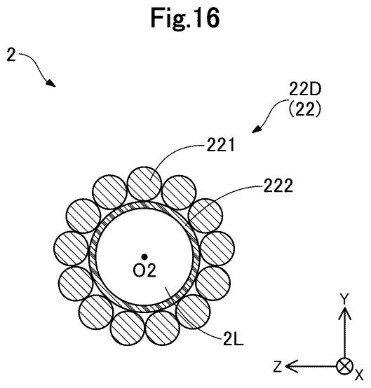

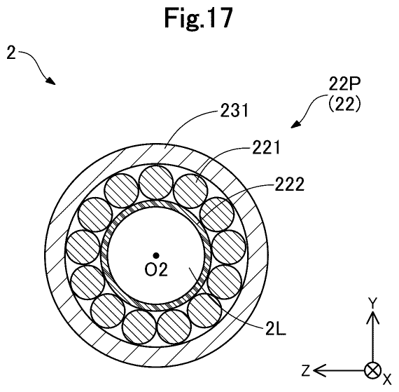

FIG. 14 is an explanatory diagram illustrating a configuration of the second catheter 2. FIG. 15 is a transverse cross-sectional view along line B1-B1 in FIG. 14. FIG. 16 is a transverse cross-sectional view along line B2-B2 in FIG. 14. FIG. 17 is a transverse cross-sectional view along line B3-B3 in FIG. 14. In FIGS. 14 to 17, the center line of the second catheter 2 is shown as the axis line O2. The axis line O2 coincides with the axis passing through the center of the second shaft 22. Hereinafter, the configuration of the second catheter 2 will be described in detail.

The puncture needle 21 is a part located at the most distal end side of the second catheter 2. As shown in FIG. 14, at the proximal end side of the puncture needle 21, the center of the puncture needle 21 coincides with the axis line 02. The distal end side of the puncture needle 21 is imparted with a curved shape that is curved in one specific direction. In the illustrated example, the one specific direction is the −Y axis direction. The part of the puncture needle 21 that is curved is also referred to as a curved portion. Because the puncture needle 21 has the curved portion, on the distal end side of the puncture needle 21, the center of the puncture needle 21 is inclined with respect to the axis line 02. The puncture needle 21 can be formed of a metal having shape memory properties. Examples of metals having shape memory properties include nickel-titanium alloys and CuZnAl alloys. The puncture needle 21 is provided in a section S21, from the distal end of the puncture needle to the distal end of the needle marker 24.

The distal end side shaft 22D is a part that is positioned further toward the proximal end side than the puncture needle 21, or in other words, between the puncture needle 21 and the proximal end side shaft 22P. As shown in FIG. 16, the distal end side shaft 22D includes a coil 221 and a tube 222.

The tube 222 maintains the liquid-tightness of the second lumen 2L of the second catheter 2. The tube 222 is a tubular body having an elongated outer shape. The distal end portion of the tube 222 is joined to the proximal end portion of the puncture needle 21. The proximal end portion of the tube 222 is joined to the distal end portion of the connector 29. The joining may be performed using metal solder, or adhesives such as epoxy adhesives and cyanoacrylic adhesives. The tube 222 can be formed of a resin having excellent chemical resistance, such as a polyimide resin.

The coil 221 imparts a predetermined rigidity and flexibility to the second shaft 22 of the second catheter 2. The coil 221 is provided in order to improve the delivery properties of the second catheter 2. The coil 221 is a multi-threaded coil formed by winding a plurality of wires into a multi-thread. The coil 221 is disposed so as to surround the outer peripheral surface of the tube 222. In the illustrated example, the inner peripheral surface of the coil 221 and the outer peripheral surface of the tube 222 are making contact with each other. The distal end portion of the coil 221 is joined to each of the tube 222 and the puncture needle 21. The proximal end portion of the coil 221 is joined to the distal end portion of the connector 29. The joining may be performed using metal solder, or adhesives such as epoxy adhesives and cyanoacrylic adhesives. The joining may be carried out by using a combination of two or more means.

The coil 221 can be formed using, for example, at least one of a stainless steel alloy, a superelastic alloy, a radiolucent alloy, and a radiopaque alloy. Examples of stainless steel alloys include SUS304 and SUS316. Examples of superelastic alloys include nickel-titanium alloy. Examples of radiolucent alloys include piano wire, nickel-chromium alloys, and cobalt alloys. Examples of radiopaque alloys include gold, platinum, tungsten, and alloys containing these elements. The 221 coil may be formed of other known materials than those mentioned above. The coil 221 may be a single-threaded coil formed by winding a single wire into a single thread. The coil 221 may be a single-threaded strand coil formed by winding a strand, in which a plurality of wires have been twisted together into a single thread. The coil 221 may be a multi-threaded strand coil formed by using a plurality of strands, in which a plurality of wires have been twisted together, and twisting the strands into a multi-thread. As shown in FIG. 14, the distal end side shaft 22D is a section S22 from the proximal end of the needle marker 24 to the boundary between the distal end side shaft 22D and the proximal end side shaft 22P.

The proximal end side shaft 22P is a part that is positioned further toward the proximal end side than the distal end side shaft 22D, or in other words, between the distal end side shaft 22D and the connector 29. As shown in FIG. 17, the proximal end side shaft 22P includes a shaft 231, a coil 221, and a tube 222. The coil 221 is the same member as the coil 221 described in FIG. 16. The tube 222 is the same member as the tube 222 described in FIG. 16.

The shaft 231 imparts a predetermined rigidity and torque transmission properties to the proximal end side shaft 22P of the second catheter 2. The shaft 231 is provided in order to improve the delivery properties of the second catheter 2. The shaft 231 is a tubular body having an elongated outer shape. The shaft 231 is disposed so as to surround the outer peripheral surface of the coil 221. In the illustrated example, the inner peripheral surface of the shaft 231 and the outer peripheral surface of the coil 221 are making contact with each other. The distal end portion of the shaft 231 is joined to a part of the coil 221. The proximal end portion of the shaft 231 is joined to the connector 29. The joining may be performed using metal solder, or adhesives such as epoxy adhesives and cyanoacrylic adhesives. The joining may be carried out by using a combination of two or more means.

The shaft 231 can be formed of a known material such as a stainless steel alloy or a superelastic alloy. Examples of stainless steel alloys include SUS302, SUS304, and SUS316. Examples of superelastic alloys include nickel-titanium alloy. As shown in FIG. 14, the proximal end side shaft 22P is a section S23 from the boundary between the distal end side shaft 22D and the proximal end side shaft 22P, to the distal end of the connector 29. In the second catheter 2 according to the present embodiment, the length of the distal end side shaft 22D and the length of the proximal end side shaft 22P in the longitudinal direction are approximately equal.

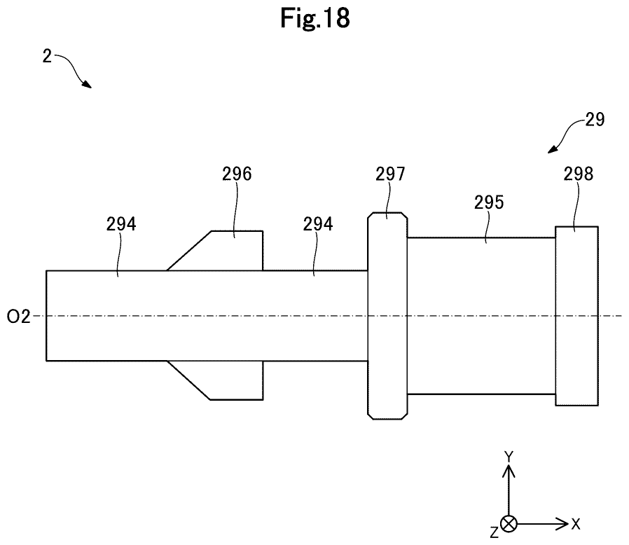

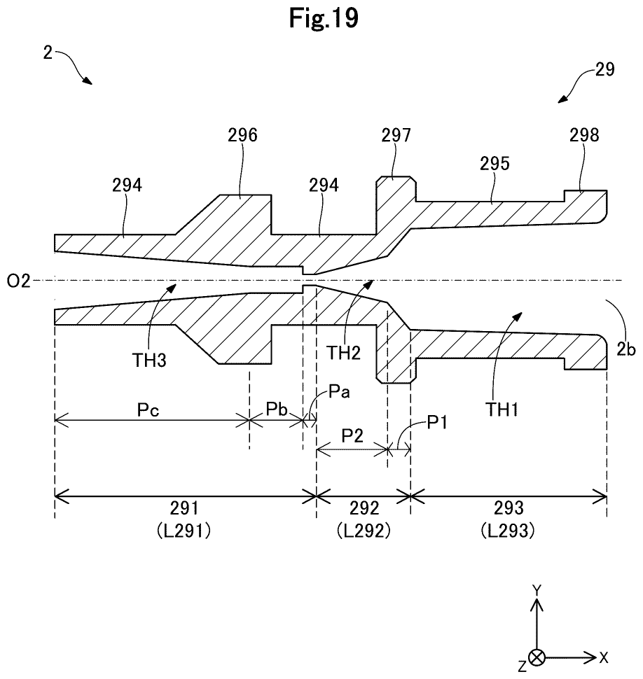

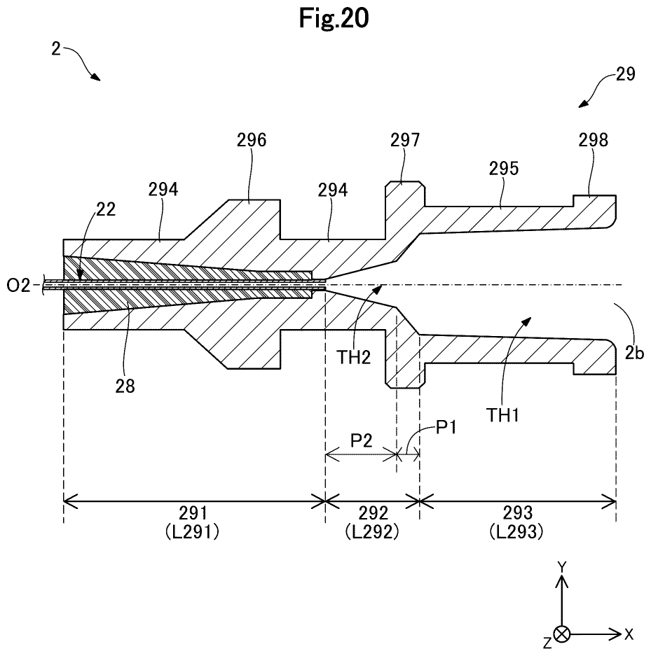

FIG. 18 is an enlarged view of the connector 29 of the second catheter 2. FIG. 19 is a longitudinal cross-sectional view of the connector 29. FIG. 20 is a longitudinal cross-sectional view of the connector 29 to which the second shaft 22 has been attached. The configuration of the connector 29 of the second catheter 2 will be described using FIGS. 18 to 20. As shown in FIG. 18, the connector 29 includes a first main body portion 294, a second main body portion 295, a wing portion 296, a first flange portion 297, and a second flange portion 298.

The first main body portion 294 is a part having a cylindrical outer shape that is disposed on the most distal end side of the connector 29. As shown in FIG. 18, the center part of the first main body portion 294 is provided with the wing portion 296. The wing portion 296 has a wing that projects in the +Y axis direction, and a wing that projects in the −Y axis direction. The second main body portion 295 is disposed further on the proximal end side than the first main body portion 294, and is a part having a cylindrical outer shape. The first flange portion 297 is a projection portion disposed on the distal end of the second main body portion 295. As shown in FIG. 19, the first flange portion 297 is an annular part that is raised toward the outside in the circumferential direction. The second flange portion 298 is a projection portion disposed on the proximal end of the second main body portion 295. As shown in FIG. 19, the second flange portion 298 is an annular part that is raised toward the outside in the circumferential direction. In the example of FIG. 19, the first main body portion 294, the second main body portion 295, the wing portion 296, the first flange portion 297, and the second flange portion 298 are integrally formed. The first main body portion 294, the second main body portion 295, the wing portion 296, the first flange portion 297, and the second flange portion 298 may each be formed as separate members.

As shown in FIG. 19, the inner side of the connector 29 is formed having, from the proximal end side toward the distal end side, a first through hole TH1, a second through hole TH2, and a third through hole TH3. The first through hole TH1, the second through hole TH2, and the third through hole TH3 are connected to each other. In other words, the second through hole TH2 is connected to the first through hole TH1. The third through hole TH3 is connected to the second through hole TH2.

In the illustrated example, the first through hole TH1 is a section from the proximal end portion of the first flange portion 297 to the proximal end of the second flange portion 298. The part of the connector 29 in which the first through hole TH1 is formed is also referred to as a proximal end portion 293. The inner diameter of the first through hole TH1 becomes slightly smaller from the proximal end side toward the distal end side. The inner diameter of the first through hole TH1 may be constant from the proximal end side toward the distal end side. A first medical device is inserted into the first through hole TH1. Examples of the first medical device include a first workhorse wire 5 and a syringe 8 described later.

In the illustrated example, the second through hole TH2 is a section from the proximal end portion of the first main body portion 294 to the proximal end portion of the first flange portion 297. The part of the connector 29 in which the second through hole TH2 is formed is also referred to as an intermediate portion 292. The intermediate portion 292 includes a first part P1 and a second part P2. The first part P1 is a part in which, on the inner peripheral surface of the second through hole TH2, the inner diameter of the second through hole TH2 becomes gradually smaller from the proximal end side toward the distal end side. The second part P2 is a part in which, on the inner peripheral surface of the second through hole TH2, the inner diameter of the second through hole TH2 becomes gradually smaller from the proximal end side toward the distal end side. The inner diameter decreases more gradually in the second part P2 than in the first part P1. The inner diameter of the distal end of the first part P1 and the inner diameter of the proximal end of the second part P2 are equal. As a result of having such a first part P1 and a second part P2, the second through hole TH2 has a trumpet shape facing the proximal end side. In the illustrated example, the length of the second part P2 is longer than the length of the first part P1 in the longitudinal direction of the connector 29.

In the illustrated example, the third through hole TH3 is a section from the distal end of the first main body portion 294 to the proximal end portion of the first main body portion 294. The part of the connector 29 in which the third through hole TH3 is formed is also referred to as a distal end portion 291. The distal end portion 291 includes a proximal end side part Pa, an intermediate part Pb, and a distal end side part Pc. The proximal end side part Pa is a part of the inner peripheral surface of the third through hole TH3 having a substantially constant inner diameter. The inner diameter of the proximal end of the proximal end side part Pa and the inner diameter of the distal end of the second part P2 are equal. The intermediate part Pb is a part of the inner peripheral surface of the third through hole TH3 having a substantially constant inner diameter that is larger than that of the proximal end side part Pa. A step is formed between the intermediate part Pb and the proximal end side part Pa. The distal end side part Pc is a part in which, on the inner peripheral surface of the third through hole TH3, the inner diameter of the third through hole TH3 becomes gradually larger from the proximal end side toward the distal end side. The inner diameter of the proximal end of the distal end side part Pc and the inner diameter of the intermediate part Pb are equal. In the illustrated example, in the longitudinal direction of the connector 29, the length of the proximal end side part Pa is the shortest, and the length of the distal end side part Pc is the longest. A second medical device is inserted into the third through hole TH3. Examples of the second medical device include the second shaft 22 described in FIG. 20.

As shown in FIG. 19, in the longitudinal direction of the connector 29, the length L 292 of the intermediate portion 292 is shorter than the length L 291 of the distal end portion 291, and is shorter than the length L293 of the proximal end portion 293. The length L 293 of the proximal end portion 293 is shorter than the length L 291 of the distal end portion 291. The length L 293 of the proximal end portion 293 may be longer than the length L 291 of the distal end portion 291. The length L293 of the proximal end portion 293 and the length L291 of the distal end portion 291 may be the same.

As shown in FIG. 20, the proximal end portion of the second shaft 22 is inserted into the third through hole TH3 of the connector 29, and is fixed thereto by a bonding agent 28. As a result, the first through hole TH1 and the second through hole TH2 form a portion of the second lumen 2L of the second catheter 2.

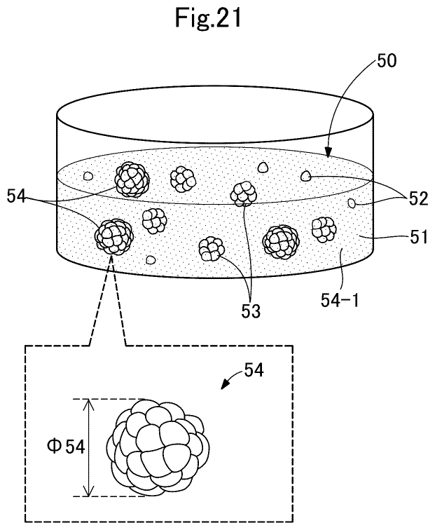

FIG. 21 is a diagram describing a drug solution 50. In the example of FIG. 21, the drug solution 50 includes a solvent 51, single cells 52, cell clusters 53, organoids 54, and chemical compounds 54-1. In the following description, the term “cells” refers to any one of pluripotent stem cells, cells derived from pluripotent stem cells, cardiomyocytes, and mesenchymal stem cells. Pluripotent stem cells refer to cells that have the ability to differentiate into various types of cells. Examples of pluripotent stem cells include iPS cells. Cells derived from pluripotent stem cells refer to cells differentiated from pluripotent stem cells. Examples of cells derived from pluripotent stem cells include iPS cell-derived cardiomyocytes. Cardiomyocytes refer to cells that constitute the myocardium. Mesenchymal stem cells refer to stem cells that are present in the body, and have the ability to differentiate into bone, cartilage, blood vessels, myocardium cells, and the like. Mesenchymal stem cells are also referred to as M SC. Examples of the low molecular weight compound 54-1 include ONO-1301, and a sustained release formulation of ONO-1301 (Y S-1402), which have both prostaglandin I2 receptor agonism and thromboxane A2 synthase inhibitory activity.

The single cells 52 are individual cells. The cells may be of a single cell type or of a plurality of cell types. A single cell type refers to cells that are all of a single type. A plurality of cell types refers to a plurality of types of cells. The single cells 52 being of a single cell type refers to the fact that the plurality of single cells 52 contained in the drug solution 50 are each the same type of cell among the four types of cells mentioned above (pluripotent stem cells, cells derived from pluripotent stem cells, cardiomyocytes, and mesenchymal stem cells). The single cells 52 being of a plurality of cell types refers to the fact that the plurality of single cells 52 contained in the drug solution 50 are different types of cells that are respectively one of the four types of cells mentioned above.

The cell clusters 53 refer to clusters formed by cells aggregating together. The cell clusters are also referred to as spheres. The cell clusters 53 may be formed of a single cell type, or may be formed of a plurality of cell types. The cell clusters 53 being of a single cell type means that the cell clusters 53 are formed by aggregation of cells that are each of the same type among the four types of cells mentioned above. The cell clusters 53 being of a plurality of cell types means that the cell clusters 53 are formed by aggregation of two or more different types of cells among the four types of cells mentioned above. Examples of the cell clusters 53 include cardiomyocyte spheres.

The organoids 54 refer to three-dimensional structures that mimic a human organ or tissue. The organoids 54 have a three-dimensional structure formed by a plurality of types of single cells or cell clusters that self-organize through interactions. The organoids 54 can also be referred to as a group of single cells or cell clusters. The organoids 54 can be configured to include, for example, cardiomyocytes and mesenchymal stem cells. For example, an operator mixes cardiomyocytes and mesenchymal stem cells in a specific ratio. The operator can form the organoids 54 by culturing and organizing a mixture of cardiomyocytes and mesenchymal stem cells. An enlarged view of an organoid 54 is shown in the blow-out in FIG. 21. The outer diameter Φ54 of the organoids 54 is approximately 50 μm or more, and approximately 200 μm. The organoids 54 often have a distorted spherical shape. For this reason, the outer diameter Φ54 of the organoids 54 is the outer diameter of the part of the organoids 54 that has the largest outer diameter.

The low molecular weight compound is preferably a compound that acts on fibroblasts, vascular smooth muscle cells, vascular endothelial cells, and the like, and promotes the expression of various protective angiogenic factors such as the vascular endothelial growth factor (VEGF), hepatocyte growth factor (HGF), basic fibroblast growth factor (bFGF), and stromal cell-derived factor-1 (SDF-1), and particularly suitable are the compound ONO-1301, and its sustained release formulation Y S-1402.

As the solvent 51, for example, physiological saline or lactated Ringer's solution can be used. The drug solution 50 contains at least one of single cells 52, cell clusters 53, and organoids 54. That is, the drug solution 50 may be configured to contain only single cells 52 in the solvent 51. The drug solution 50 may be configured to contain only single cell clusters 53 in the solvent 51. The drug solution 50 may be configured to contain only organoids 54 in the solvent 51. The drug solution 50 may be configured to contain only the low molecular weight compound 54-1 in the solvent 51.

In addition to the components mentioned above, other substances may be added to the drug solution 50. Examples of other substances include human serum albumin and radiopaque components. Examples of radiopaque components include contrast agents. When a configuration is used in which the drug solution 50 contains human serum albumin, the adhesion of the single cells 52, cell clusters 53, organoids 54, and low molecular weight compound 54-1 to the inner wall of the second catheter 2 can be suppressed. Therefore, the loss of cells and the low molecular weight compound can be reduced. If the drug solution 50 contains human serum albumin, the shear stress on the cells caused by the movement of the drug solution 50 inside the second lumen 2L can be reduced. In other words, damage to the single cells 52, cell clusters 53, and organoids 54 caused by the movement of the drug solution 50 inside the second lumen 2L can be reduced. Human serum albumin is also referred to as HSA. When a configuration is used in which the drug solution 50 contains a radiopaque component, the surgeon is capable of confirming the injection effect using X-ray images during the drug solution injection treatment procedure. The amount of human serum albumin added to the drug solution 50 can be arbitrarily determined. The amount of the radiopaque component added to the drug solution 50 can be arbitrarily determined. The amount of contrast agent added to the drug solution 50 is preferably determined taking into consideration the effect on the single cells 52, the cell clusters 53, the organoids 54, and the low molecular weight compound 54-1.

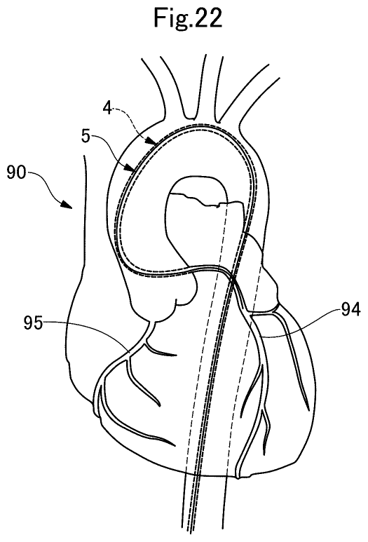

FIG. 22 is a diagram showing a state in which a drug solution injection treatment is being performed on the heart 90. Hereinafter, the surgical method of a drug solution injection treatment will be described using FIGS. 22 to 31. In the following example, a case where the surgeon accesses the myocardium from a coronary artery will be illustrated. The surgeon may access the myocardium through a coronary vein rather than a coronary artery. The drug solution injection treatment may be performed simultaneously with a percutaneous coronary intervention procedure, or may be performed independently. Percutaneous coronary intervention is also referred to as PCI.

After administering local anesthesia to the patient, the surgeon inserts a guiding catheter 4 into the artery from the wrist or groin. As shown in FIG. 22, the surgeon inserts the distal end of the guiding catheter 4 into the entrance portion of the left coronary artery 94. The surgeon allows a contrast agent to flow into the left coronary artery 94 using the guiding catheter 4, and acquires an X-ray image. The surgeon checks the acquired X-ray image and confirms that there are no problems with the left coronary artery 94, such as blockages or constrictions. As shown in FIG. 22, the surgeon delivers the first workhorse wire 5 to the treatment target site. The first workhorse wire 5 passes through a lumen inside the guiding catheter 4, and is delivered to the treatment target site inside the left coronary artery 94.

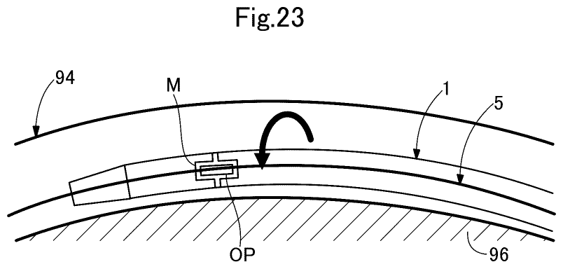

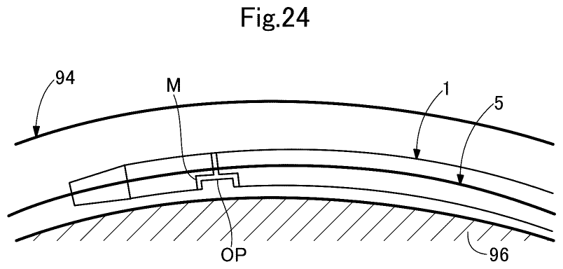

FIG. 23 is a diagram showing a state where the first catheter 1 has been delivered to a treatment target site. In FIG. 23, for convenience of the description, the device inside the first catheter 1 is also illustrated with a solid line. The same applies to FIG. 24 and subsequent diagrams. The surgeon inserts the proximal end portion of the first workhorse wire 5 inside the body into the first lumen 1L from the distal end opening la of the first catheter 1. The surgeon delivers the first catheter 1 to the treatment target site along the first workhorse wire 5. At this time, as shown in FIG. 23, the lateral opening OP of the first catheter 1 and the myocardium 96 are not aligned. As a result, the lateral opening OP of the first catheter 1 is facing an arbitrary direction.

FIG. 24 is a diagram showing a state of the first catheter 1 in which the lateral opening OP is facing the myocardium 96. After delivery of the first catheter 1, the surgeon aligns the lateral opening OP of the first catheter 1 and the myocardium 96. Specifically, while viewing the image of the marker M under an X-ray image, the surgeon rotates the first catheter 1 using the shape of the marker M as a guide, so that the lateral opening OP faces the myocardium 96 side. As a result, as shown in FIG. 24, the lateral opening OP of the first catheter 1 and the myocardium 96 are aligned.

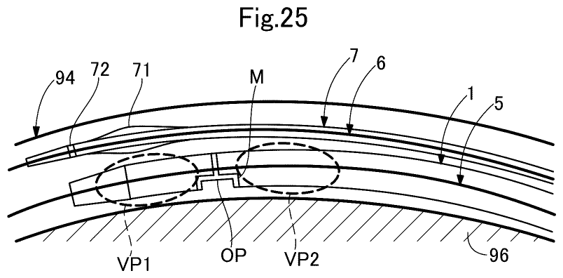

FIG. 25 is a diagram showing a state where the balloon catheter 7 is being delivered. The surgeon delivers a second workhorse wire 6, which is different from the first workhorse wire 5, to the treatment target site. The surgeon delivers the balloon catheter 7 to the treatment target site along the second workhorse wire 6. The balloon catheter 7 includes a balloon 71 and a marker 72. The balloon 71 is a film-like member that expands due to supply of a fluid, and contracts due to discharge of the fluid. The marker 72 is a radiopaque marker for grasping the position of the balloon 71 under an X-ray image. The surgeon disposes the balloon 71 in one of a first position VP1 and a second position V P2 while viewing the image of the marker 72 under an X-ray image. The first position VP1 is located near the lateral opening OP of the first catheter 1 in the extending direction of the left coronary artery 94, and is a position further on the distal end side than the lateral opening OP. The second position VP2 is located near the lateral opening OP of the first catheter 1 in the extending direction of the left coronary artery 94, and is a position further on the proximal end side than the lateral opening OP. In the example of FIG. 25, the balloon 71 is disposed in the first position VP1. At this point, the surgeon does not yet inflate the balloon 71. In a case where the surgeon determines that piercing assistance from the balloon catheter 7 is not necessary, or that the inner diameter of the left coronary artery 94 is not sufficient to accommodate the parallel passage of the catheter, the surgeon may omit the placement procedure of the balloon catheter 7.

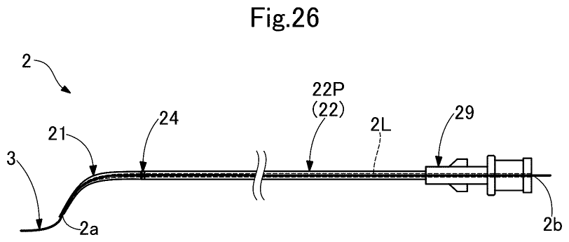

FIG. 26 is a diagram showing a state where the stylet wire 3 is being inserted through the second catheter 2. In FIG. 26, for convenience of the description, the device inside the second catheter 2 is also illustrated with a solid line. The same applies to FIG. 26 and subsequent diagrams. The surgeon inserts the stylet wire 3 into the second lumen 2L from the proximal end opening 2b of the second catheter 2, and draws the stylet wire 3 out from the distal end opening la of the second catheter 2. As a result, as shown in FIG. 26, the distal end portion of the stylet wire 3 projects out from the distal end of the second catheter 2, that is, projects out from the distal end of the puncture needle 21. The state of FIG. 26 is also referred to as an inserted state of the stylet wire 3. The projection length of the stylet wire 3 from the distal end of the second catheter 2 may be arbitrarily determined. In this way, the surgeon inserts the stylet wire 3 into the second catheter 2 in advance, prior to inserting the second catheter 2 into the first catheter 1.

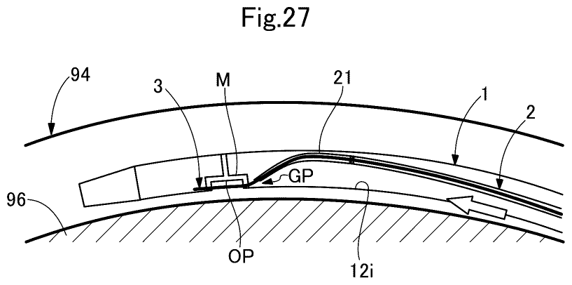

FIG. 27 is a diagram showing a state where the second catheter 2 is being delivered. The surgeon inserts the second catheter 2, to which the stylet wire 3 has been inserted, into the first lumen 1L from the proximal end opening 1b of the first catheter 1. The surgeon pushes and advances the second catheter 2 inside the first lumen 1L of the first catheter 1, and delivers the second catheter 2 until the distal end of the second catheter 2 is positioned near the lateral opening OP of the first catheter 1. In the illustrated example, the distal end of the second catheter 2 is positioned short of the lateral opening OP. The surgeon is capable of grasping the distal end position of the second catheter 2 from the image of the puncture needle 21 under an X-ray image. As shown in FIG. 27, when the second catheter 2 is delivered, the stylet wire 3 projecting from the distal end of the second catheter 2 holds up the puncture needle 21 of the second catheter 2. In other words, a gap GP occurs between the stylet wire 3 and an inner peripheral surface 12i of the first catheter 1 due to the rigidity of the stylet wire 3. As a result of the gap GP, contact of the puncture needle 21 of the second catheter 2 with the inner peripheral surface 12i of the first catheter 1 is suppressed. As a result, damage to the second catheter 2 and damage to the first catheter 1 can be suppressed when the second catheter 2 is delivered. As shown in FIG. 27, the curved shape of the puncture needle 21 of the second catheter 2 is gently corrected due to the insertion of the stylet wire 3 and the inner peripheral surface 12i of the first catheter 1.

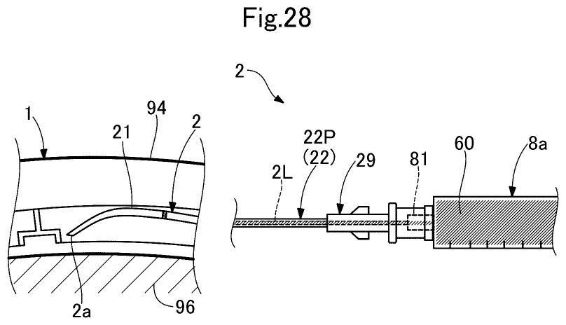

FIG. 28 is a diagram showing a state where the buffer solution 60 is being injected into the second catheter 2. The left side of FIG. 28 illustrates a part of the first catheter 1 and the second catheter 2 inside the left coronary artery 94. The right side of FIG. 28 illustrates the connector 29 of the second catheter 2, which is outside the body. After delivering the second catheter 2, the surgeon removes the stylet wire 3 from the second catheter 2. Specifically, the surgeon removes the stylet wire 3 inside the second catheter 2 by pulling out the proximal end portion of the stylet wire 3, which is outside the body, toward the hand side. After removing the stylet wire 3, the surgeon prepares a syringe 8a filled with the buffer solution 60. As shown in FIG. 28, the surgeon inserts a cylinder tip 81 of the syringe 8a into the first through hole TH1 of the connector 29 of the second catheter 2. The surgeon pushes the plunger of the syringe 8a. As a result, the buffer solution 60 inside the syringe 8a is supplied to the second lumen 2L of the second catheter 2. The surgeon continues to supply the buffer solution 60 until the second lumen 2L of the second catheter 2 is filled with the buffer solution 60. An arbitrary liquid can be used as the buffer solution 60. For example, physiological saline can be used as the buffer solution 60.

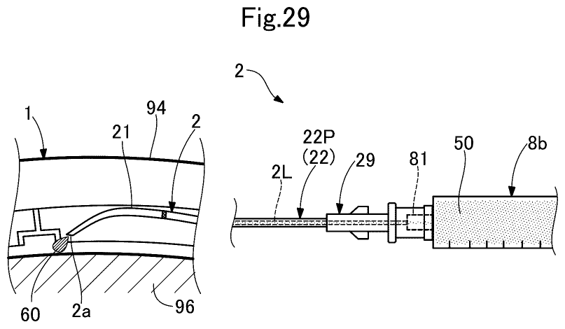

FIG. 29 is a diagram showing a state where the drug solution 50 is being injected into the second catheter 2. The left side of FIG. 29 illustrates a part of the first catheter 1 and the second catheter 2 inside the left coronary artery 94. The right side of FIG. 29 illustrates the connector 29 of the second catheter 2, which is outside the body. After filling the second catheter 2 with the buffer solution 60, the surgeon detaches the syringe 8a, and prepares a syringe 8b filled with the drug solution 50. As shown in FIG. 29, the surgeon inserts a cylinder tip 81 of the syringe 8b into the first through hole TH1 of the connector 29 of the second catheter 2. The surgeon pushes the plunger of the syringe 8b. As a result, the drug solution 50 inside the syringe 8b is supplied to the second lumen 2L of the second catheter 2. Because the second lumen 2L of the second catheter 2 is already filled with the buffer solution 60, as the drug solution 50 is supplied, the buffer solution 60 that is pushed out by the drug solution 50 is discharged from the distal end opening 2a of the second catheter 2. That is, the buffer solution 60 inside the second lumen 2L of the second catheter 2 is replaced by the drug solution 50. The surgeon continues to supply the drug solution 50 until the buffer solution 60 inside the second lumen 2L is completely replaced by the drug solution 50, and the second lumen 2L is filled with the drug solution 50.

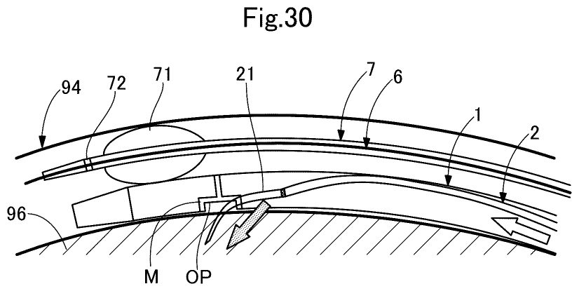

FIG. 30 is a diagram showing a state where the myocardium 96 is being pierced. After filling the inside of the second catheter 2 with the drug solution 50, the surgeon once again confirms that the lateral opening OP of the first catheter 1 is facing the myocardium 96 side by checking the marker M under an X-ray image. After the confirmation, the surgeon pushes the second catheter 2 in the direction of the white arrow while keeping the position of the first catheter 1 fixed. As a result, the surgeon is capable of projecting the puncture needle 21 of the second catheter 2 from the lateral opening OP of the first catheter 1, and piercing the myocardium 96 with the puncture needle 21. The surgeon pushes and advances the distal end of the puncture needle 21 to the target position inside the myocardium 96 while confirming the sense of resistance felt by the hands due to the piercing, and the image of the puncture needle 21 under an X-ray image. The target position is the position inside the myocardium 96 at which the surgeon intends to inject the drug solution 50.

In a case where the surgeon determines that assistance is needed to perform the piercing, the balloon 71 may be expanded prior to pushing in the second catheter 2. Examples of cases where assistance to perform the piercing is necessary include cases where it is expected that it will be difficult to pierce the myocardium 96 using the puncture needle 21 due to resistance from the myocardium 96, and cases where it is expected that the piercing depth into the myocardium 96 will be insufficient. In this case, the surgeon supplies a fluid for expanding the balloon 71 to the balloon catheter 7. As shown in FIG. 30, the balloon 71 expands toward the outside in the radial direction with the supply of fluid. The first catheter 1 is pressed against the inner wall of the blood vessel of the left coronary artery 94 by the expanded balloon 71, which fixes the position of the first catheter 1. As a result, the surgeon is capable of smoothly piercing the myocardium 96 even when there is a resistive force from the myocardium 96.

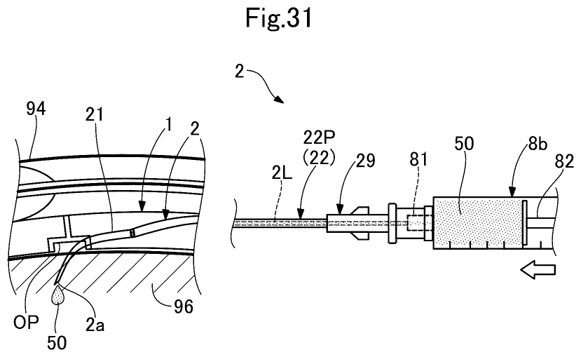

FIG. 31 is a diagram showing a state where the drug solution 50 is being injected into the myocardium 96. The left side of FIG. 31 illustrates a part of the first catheter 1 and the second catheter 2 inside the left coronary artery 94. The right side of FIG. 31 illustrates the connector 29 of the second catheter 2, which is outside the body. After performing the piercing using the puncture needle 21 to the target position, the surgeon injects the drug solution 50 into the tissue of the myocardium 96 by pushing the plunger 82 of the syringe 8b. When the drug solution 50 contains a radiopaque component, the surgeon can inject the drug solution 50 while observing the state of the injection of the drug solution 50 into the myocardium 96 under an X-ray image.

After completing the injection of the drug solution 50, the surgeon slowly pulls the second catheter 2 toward the hand side. As a result, the puncture needle 21 of the second catheter 2 is pulled out from the myocardium 96. As a result of the surgeon further pulling the second catheter 2 toward the hand side, the puncture needle 21 of the second catheter 2 can be accommodated inside the first lumen 1L of the first catheter 1. At this time, as shown in FIG. 28, the surgeon preferably positions the distal end of the second catheter 2 short of the lateral opening OP of the first catheter 1. The surgeon causes the balloon 71 to deflate as a result of suction of the fluid from the balloon catheter 7, and releases the fixed state of the first catheter 1.

The surgeon moves the first catheter 1 inside the left coronary artery 94, and repeats the alignment described using FIG. 24, the delivery of the balloon catheter 7 described using FIG. 25, the piercing of the myocardium 96 described using FIG. 30, and the injection of the drug solution 50 described using FIG. 31 a planned number of times. In this way, by moving the first catheter 1 to different positions in the left coronary artery 94 and then repeating the procedure, the drug solution 50 can be injected to different positions of the myocardium 96. Similarly, with respect to the right coronary artery 95, the surgeon may inject the drug solution 50 into the myocardium 96 via the right coronary artery 95. The surgeon may inject the drug solution 50 from only the right coronary artery 95 instead of the left coronary artery 94, or may inject the drug solution 50 from both the left coronary artery 94 and the right coronary artery 95.

The surgeon confirms whether or not injection of the drug solution 50 into the treatment target site at predetermined locations, and delivery of a predetermined amount of the drug solution 50, has been completed. In order to obtain the effects of the drug solution injection treatment, it is preferable that the number of cells contained in the predetermined amount of the drug solution 50 is 100 million cells or more per patient. Furthermore, in order to obtain the effects of the drug solution injection treatment, it is preferable that the amount of the low molecular weight compound contained in the predetermined amount of the drug solution 50 is 0.03 to 0.3 mg/kg weight per patient. After the confirmation, the surgeon removes all of the devices except the guiding catheter 4 from the left coronary artery 94. The surgeon allows a contrast agent to flow into the left coronary artery 94 using the guiding catheter 4, and acquires an X-ray image. The surgeon confirms the X-ray image to check that there is no leakage of blood from the left coronary artery 94. In a case where the drug solution 50 is injected via the right coronary artery 95, the surgeon similarly confirms the X-ray image for the right coronary artery 95, and checks that there is no leakage of blood from the right coronary artery 95. If there is no leakage of blood from the left coronary artery 94 and the right coronary artery 95, the surgeon removes the guiding catheter 4, and ends the procedure.

As described above, because the first catheter 1 includes the radiopaque marker M, which indicates the position of the lateral opening OP, the surgeon is capable of easily matching the positions of the lateral opening OP and the myocardium 96 in the circumferential direction by confirming the position of the marker M under an X-ray image. Then, because the surgeon directly injects the drug solution 50 containing therapeutic cells into the myocardium tissue from the puncture needle 21 projecting from the lateral opening OP, the treatment effects due to the drug solution 50 can be improved compared to a case where the drug solution 50 is administered by other means. As a result, the efficiency of the drug solution injection treatment procedure can be improved. According to such a drug solution injection treatment, the physical burden on the patient can be reduced compared to surgical treatment methods, and improvements in the QOL after treatment can be expected. QOL is also referred to as quality of life.

The puncture needle used in the drug solution injection treatment often has a curved shape. This is because the puncture needle is projected from a coronary artery or a coronary vein into the myocardium. When a puncture needle having such a curved shape is installed in a catheter, the central axis of the catheter becomes offset, resulting in poor rotation control. According to the present method, the surgeon can deliver the first catheter 1 to the treatment target site, and then deliver the second catheter 2 to the treatment target site inside the first lumen 1L of the first catheter 1. Because the first catheter 1 does not include the puncture needle and has excellent rotation control, the first catheter 1 can be smoothly delivered to the treatment target site. Because the second catheter 2 is pushed and advanced inside the first catheter 1, whose path has already been secured, the surgeon can smoothly deliver the second catheter 2 to the treatment target site. As a result, the efficiency of the drug solution injection treatment procedure can be further improved. In addition, by using the first catheter 1 and the second catheter 2 configured as separate devices, the outer diameters of the first catheter 1 and the second catheter 2 can be made smaller compared to a device provided with a multi-lumen catheter. As a result, it becomes possible to insert the device through narrow blood vessels of the coronary artery or the coronary vein, and the range of applications of the present method can be expanded.

Further, before inserting the second catheter 2 into the first catheter 1, the stylet wire 3 is inserted into the second catheter 2 so that the distal end portion of the stylet wire 3 projects from the distal end of the second catheter 2. As a result, when the second catheter 2 is delivered, the stylet wire 3 is capable of functioning as a cushioning material. Specifically, the stylet wire 3 projecting from the distal end of the second catheter 2 is capable of preventing the puncture needle 21 at the distal end of the second catheter 2 from making contact with the inner peripheral surface 12i of the first catheter 1. As a result, damage to the puncture needle 21 and damage to the first catheter 1 can be suppressed at the time of delivery of the second catheter 2.

In addition, after delivery of the second catheter 2, by removing the stylet wire 3 from the second catheter 2, the bent shape of the puncture needle 21, which had become more gentle due to insertion of the stylet wire 3, can be returned to the original bent shape. As a result, the surgeon can easily project the puncture needle 21 from the lateral opening OP of the first catheter 1 toward the outside.

In addition, after removal of the stylet wire 3, because the surgeon fills the second catheter 2 with the buffer solution 60, the air inside the second lumen 2L of the second catheter 2 can be removed due to the buffer solution 60. Also, the surgeon can prevent damage to the cells inside the drug solution 50 by wetting the inside of the second lumen 2L in advance with the buffer solution 60, and then replacing the buffer solution 60 with the drug solution 50.

Further, because the surgeon uses the balloon catheter 7 to press the first catheter 1 against the inner wall of a blood vessel, it is possible to prevent the displacement of the first catheter 1 at the time of performing piercing with the puncture needle 21. Displacement of the first catheter 1 at the time of performing the piercing refers to movement of the first catheter 1 in a direction away from the myocardium 96. In addition, the surgeon disposes the balloon 71 of the balloon catheter 7 near the lateral opening OP and in a position not overlapping the lateral opening OP. As a result, at the time of performing the piercing, the first catheter 1 is supported by the balloon 71, and the lateral opening OP of the first catheter 1 can be prevented from becoming blocked by the balloon 71.

Also, the drug solution 50 includes at least one of single cells, cell clusters, groups of single cells or cell clusters, and chemical compounds. Moreover, as the therapeutic cells contained in the drug solution 50, pluripotent stem cells, cells derived from pluripotent stem cells, cardiomyocytes, and mesenchymal stem cells can be utilized. Further, in a case where the drug solution 50 contains organoids, the treatment effects are improved. In addition, as the low molecular weight compound contained in the drug solution 50, ONO-1301 and a sustained release formulation of ONO-1301 (YS-1402), which have both prostaglandin I2 receptor agonism and thromboxane A2 synthase inhibitory activity, can be utilized.

Further, because the medical system 1000 includes the drug solution 50, and the puncture needle 21 for injecting the drug solution 50 into the myocardium, a simple medical system that can be used in drug solution injection treatments can be provided.

In addition, according to the medical system 1000, the intermediate portion 292 of the connector 29 of the second catheter 2 includes the first part P1 and the second part P2, in which the diameter of the second through hole TH2 becomes smaller from the proximal end side toward the distal end side. As a result, the volume inside the second through hole TH2 can be reduced, and the amount of the drug solution 50 that remains inside the second through hole TH2 after the drug solution injection treatment can be reduced. Consequently, waste of the drug solution 50 can be suppressed. The second part P2, which has a diameter that becomes smaller from the proximal end side toward the distal end side more gradually than the first part, is provided further toward the distal end side than the first part P1. As a result, the pressure at the time of injection when the drug solution 50 is injected from the syringe 8b, which serves as the first medical device, can be dispersed.

In addition, according to the medical system 1000, the marker M of the first catheter 1 includes the first marker portion M1, which surrounds the outer edge of the lateral opening OP, and the second marker portion M2, which is provided in a different position to the first marker portion M1. As a result, under an X-ray image, it is possible for the surgeon to easily grasp the orientation of the lateral opening OP by the change in the shape of the marker M accompanying the change in the positional relationship between the first marker portion M1 and the second marker portion M2 when the first catheter 1 is rotated around the central axis of the first shaft 12. As a result, the efficiency of the procedure can be improved. Because the second marker portion M2 is provided in the residual region in which the first marker portion M1 is not provided, the occurrence of kinks in the first catheter 1 due to stress concentration caused by the stress difference between the lateral opening OP and the first marker portion M1 can be suppressed on the lateral surface of the section in the longitudinal direction of the first shaft 12 in which the lateral opening OP is provided. As a result, the safety of the procedure can be improved.

Also, according to the medical system 1000, when the marker M is viewed under an X-ray image from the side of the direction perpendicular to the central axis of the first shaft 12 and the opening direction of the lateral opening OP, the length of the first marker portion M1 along the opening direction tends to be relatively short, and can be difficult to visually recognize. According to the first catheter 1, because the length of the second marker portion M2 along the opening direction is longer than length of the first marker portion M1 along the opening direction, the surgeon can more easily grasp the orientation of the lateral opening OP. As a result, the surgeon can more easily adjust the orientation of the lateral opening OP to the desired orientation, and the efficiency of the procedure can be further improved.

Further, according to the medical system 1000, because the shape of the lateral opening OP of the first catheter 1 is a shape that does not include corners, it is possible to prevent the lateral opening OP from becoming caught on the inner wall of the blood vessel when the first catheter 1 is being inserted into the coronary artery or the coronary vein. As a result, damage to the inner wall of the blood vessel can be suppressed, and the safety of the procedure can be further improved.

In addition, according to the medical system 1000, in the first catheter 1, the length of the first marker portion M1 along the longitudinal direction of the first shaft 12 is longer than the length of the second marker portion M2. As a result, the surgeon can easily distinguish and visually recognize the first marker portion M1 and the second marker portion M2. Also, because the second marker portion M2 extends in the circumferential direction, it is possible for the surgeon to more easily grasp the orientation of the lateral opening OP under an X-ray image when the first catheter 1 is rotated around the central axis of the first shaft 12. Further, both ends of the second marker portion M2 that extend in the circumferential direction are connected to the first marker portion M1. As a result, the occurrence of kinks in the first catheter 1 due to stress concentration caused by the stress difference between the lateral opening OP and the first marker portion M1 can be further suppressed on the lateral surface of the section in the longitudinal direction of the first shaft 12 in which the lateral opening OP is provided.

### Second Embodiment

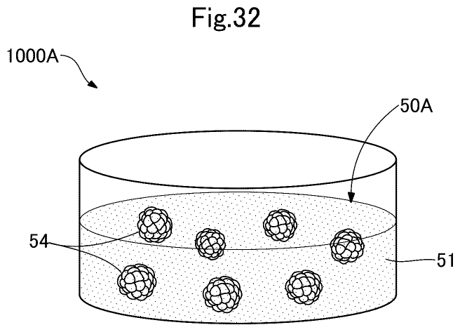

FIG. 32 is a diagram describing a drug solution 50A according to a second embodiment. A medical system 1000A according to the second embodiment includes a drug solution 50A instead of the drug solution 50 in the configuration described in the first embodiment. The drug solution 50A may be configured to contain only organoids 54 in the solvent 51.

In this way, the configuration of the drug solution 50A can be modified in various ways, and may be configured to include only the organoids 54. The configuration of the organoids 54 is as described in FIG. 21. The drug solution 50A may be configured to contain only the single cells 52 described in FIG. 21 in the solvent 51. The drug solution 50A may be configured to contain only the cell clusters 53 described in FIG. 21 in the solvent 51. The drug solution 50A may be configured to contain only the low molecular weight compound 54-1 described in FIG. 21 in the solvent 51. The same effects as those obtained in the first embodiment described above can also be obtained in the second embodiment.

### Third Embodiment

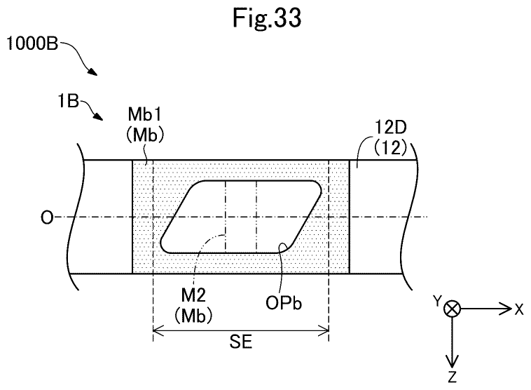

FIG. 33 is a diagram showing the shape of a marker Mb when a first catheter 1B according to a third embodiment is viewed from the −Y axis direction. The medical system 100B according to the third embodiment includes a first catheter 1B instead of the first catheter 1 in the configuration described in the first embodiment. Of the configuration described in the first embodiment, the first catheter 1B includes a lateral opening OPb instead of the lateral opening OP, and includes a marker Mb instead of the marker M.

As shown in FIG. 33, the shape of the lateral opening OPb when viewed from the opening direction is a shape that does not include corners. Specifically, the shape of the lateral opening OPb when viewed from the opening direction side is a substantially parallelogram shape in which the four corners are rounded. That is, the shape of the lateral opening OPb when viewed from the opening direction is a shape that is asymmetric with respect to the axis line O. The marker M b includes a first marker portion Mb1 and a second marker portion M2. The first marker portion Mb1 surrounds the outer edge of the lateral opening OPb, which has a substantially parallelogram shape, on the lateral surface of the distal end side shaft 12D. That is, when the lateral opening OPb is viewed from the opening direction side, the first marker portion Mb1 has a shape that is asymmetric with respect to the axis line O. The second marker portion M2, as in the first embodiment, extends in the circumferential direction in the residual region R. Both ends of the second marker portion M2 that extend in the circumferential direction are connected to the first marker portion Mb1.

In this way, various modifications are possible to the configuration of the first catheter 1B, and when the lateral opening OPb is viewed from the opening direction side, the first marker portion Mb1 having a shape that is asymmetric with respect to the axis line O may be provided. Effects equivalent to those described in the first embodiment can also be obtained by the medical system 1000B according to the third embodiment described above. According to the medical system 1000B of the third embodiment, because the shape of the marker M b when viewed under an X-ray image from the opening direction side of the first catheter 1B is clearly different to the shape of the marker Mb when viewed from the opposite direction side to the opening direction side, the surgeon can easily grasp the orientation of the lateral opening OPb. As a result, the surgeon can more easily adjust the orientation of the lateral opening OPb to the desired orientation, and the efficiency of the procedure can be further improved.

### Modification of Embodiments

The present disclosure is not limited to the embodiments described above, and may be implemented in various forms without departing from the gist of the present disclosure. For example, the following modifications are also possible.

### First Modification

Examples of the configurations of the medical systems 1000, 1000A, and 1000B have been described in the first to third embodiments above. Various modifications are possible to the configurations of the medical systems 1000, 1000A, and 1000B. For example, the stylet wire 3 may be omitted. For example, the drug solution 50 may be omitted. For example, the medical system 1000 may be configured by including other devices not mentioned above. Examples of other devices include the guiding catheter 4, the balloon catheter 7, and the syringe 8.

### Second Modification

Examples of the configurations of the first catheters 1 and 1B have been described in the first to third embodiments above. Various modifications are possible to the configurations of the first catheters 1 and 1B. For example, the first shaft 12 is not limited to the configuration described using FIGS. 2 to 4, and various configurations may be adopted. For example, the first shaft 12 may be configured by omitting at least some of the inner layer 13, the outer layer 14, the coil 15, the reinforcing body 16, and the coating layer 17. For example, the first shaft 12 may not have the distal end side shaft 12D and the proximal end side shaft 12P, and have the same configuration from the distal end to the proximal end. For example, the first catheter 1 does not have to include the marker M. For example, the marker M may not have the first marker portion M1 and the second marker portion M2, and have a simple, annular shape.

### Third Modification

An example of the configuration of the second catheters 2 has been described in the first to third embodiments above. Various modifications are possible to the configuration of the second catheters 2. For example, the stylet wire 3 may be omitted. For example, the drug solution 50 may be omitted. For example, the second shaft 22 is not limited to the configuration described using FIGS. 15 to 17, and various configurations may be adopted. For example, the second shaft 22 may be configured by omitting at least some of the coil 221, the tube 222, and the shaft 223. For example, the second shaft 22 may not have the distal end side shaft 22D and the proximal end side shaft 22P, and may have the same configuration from the distal end to the proximal end. For example, the second catheter 2 does not have to include the needle marker 24. For example, the connector 29 of the second catheter 2 is not limited to the configuration described in FIGS. 18 to 20. For example, the second through hole TH2 of the connector 29 does not have to include the first part P1 and the second part P2. For example, the connector 29 may have the same configuration as the catheter connector 19 of the first catheter 1.

### Fourth Modification

In the first to third embodiments above, an example of the surgical method of a drug solution injection treatment has been described. Various modifications can be made to the surgical method of a drug solution injection treatment. For example, the procedure in which the surgeon delivers the balloon catheter 7 and the second workhorse wire 6 may be omitted. For example, the procedure in which the surgeon inserts the stylet wire 3 through the second catheter 2 may be omitted. For example, the procedure in which the surgeon injects the buffer solution 60 into the second catheter 2 may be omitted. For example, the surgeon may use a device other than the syringe 8 to inject the buffer solution 60 or the drug solution 50. For example, the surgeon may further perform procedures not mentioned above in the surgical method of a drug solution injection treatment. For example, the surgeon may perform a PCI procedure prior to the procedure described above.

### Fifth Modification

The configurations of the first to third embodiments described above, and the configurations of the first to fourth modifications described above may be combined as appropriate. For example, the medical system 1000 can be configured by combining the drug solution 50A described in the second embodiment, and the first catheter 1B described in the third embodiment.

The present aspect has been described above based on the embodiments and the modifications. The embodiments of the aspect described above are intended to facilitate understanding of the present aspect, and do not limit the present aspect. The present aspect may be modified and improved without departing from the gist and the scope of the claims, and the present aspect includes equivalents thereof. If the technical features are not described as essential in the present specification, the technical features may be appropriately removed.

What is claimed is:

1. A surgical method for delivering a drug solution to myocardium, comprising:
inserting a catheter into at least one of a coronary artery and a coronary vein, the catheter having
a lumen,
a lateral opening connecting the lumen and outside of the catheter, and
a radiopaque marker indicating a position of the lateral opening;
rotating the catheter while observing the radiopaque marker such that an orientation of the lateral opening becomes a predetermined orientation with respect to a treatment target site;
projecting a puncture needle through the lateral opening;
piercing a myocardium of the treatment target site with the puncture needle; and
injecting a drug solution containing therapeutic cells or chemical compounds from the puncture needle into the myocardium.

2. The surgical method according to claim 1, wherein
the catheter is a first catheter, and the lumen is a first lumen, and
the puncture needle is provided on a distal end of a second catheter that is inserted into the first lumen of the first catheter, the second catheter having a second lumen.

3. The surgical method according to claim 2, wherein
before the second catheter is inserted into the first catheter, a stylet wire is inserted into the second lumen of the second catheter, and a distal end portion of the stylet wire is projected out from the distal end of the second catheter; and
the second catheter, into which the stylet wire has been inserted, is inserted into the first lumen of the first catheter so that the distal end of the second catheter is advanced, inside the first lumen, to a position near the lateral opening of the first catheter.

4. The surgical method according to claim 3, wherein
the stylet wire is removed from the second catheter after the second catheter has been advanced to the position near the lateral opening.

5. The surgical method according to claim 4, wherein
the second lumen of the second catheter is filled with a buffer solution after removal of the stylet wire.

6. The surgical method according to claim 5, wherein
the drug solution is injected into the second lumen of the second catheter after being filled with the buffer solution, and the buffer solution inside the second lumen is replaced by the drug solution.

7. The surgical method according to claim 2, wherein
a distal end portion of the second catheter is projected out from the lateral opening of the first catheter, and
the drug solution is injected into the myocardium by piercing the myocardium with the puncture needle of the second catheter.

8. The surgical method according to claim 1, wherein
the drug solution is injected into the myocardium in a state where the catheter is pushed against an inner wall of a blood vessel in a fixed position due to inflation of a balloon of a balloon catheter.

9. The surgical method according to claim 8, wherein
the balloon of the balloon catheter is arranged, in an extending direction of the blood vessel, near the lateral opening of the catheter, at either a position further on a distal end side than the lateral opening, or a position further on a proximal end side than the lateral opening.

10. The surgical method according to claim 1, wherein
the drug solution contains at least one of single cells, cell clusters, groups of single cells or cell clusters, and chemical compounds; and
the cells include at least one of a single cell type or a plurality of cell types.

11. The surgical method according to claim 10, wherein
the cells are any one of pluripotent stem cells, cells derived from pluripotent stem cells, cardiomyocytes, and mesenchymal stem cells.
